(19) United States
(12) Patent Application Publication
(10) Pub. No.: US 2025/0164234 A1
(43) Pub. Date: May 22, 2025

(54) Line-field OCT System with Multi Transverse Mode Laser

(71) Applicant: KineoLabs, Inc., Billerica, MA (US)

(72) Inventors: Yisi Liu, Winchester, MA (US); Walid Atia, Jamaica Plain, MA (US)

(21) Appl. No.: 18/950,320

(22) Filed: Nov. 18, 2024

Related U.S. Application Data
(60) Provisional application No. 63/600,099, filed on Nov. 17, 2023.
(60) Provisional application No. 63/652,442, filed on May 28, 2024.

(51) Int. Cl. G01B 9/02091; G01B 9/02; G01B 9/02015

(52) U.S. Cl. CPC G01B 9/02091; G01B 9/02015; G01B 9/02037; G01B 9/02041; G01B 9/02051; G01B 9/02083; G01B 2290/15

(57) ABSTRACT

A line-field swept-source optical coherence tomography (OCT) system features a cat's-eye tunable laser that is free-space coupled to an interferometer. The laser employs a single angled facet (SAF) edge-emitting gain chip producing a beam with multiple spatial modes. By preserving these higher-order modes through free-space coupling—avoiding single-mode fiber—the system generates a line with a more uniform, top-hat intensity profile when projected onto a sample, such as a patient's retina. This profile mitigates the Gaussian power roll-off typically associated with single spatial mode beams, ensuring adequate signal-to-noise ratio across the line while adhering to optical safety limits. The laser cavity includes a thin-film interference bandpass filter mounted on an angle control actuator, allowing for wavelength sweeping by tilt-tuning the filter. The system integrates a line-scan sensor and is designed for manufacturability, offering improved imaging quality for ophthalmic diagnostics and other applications requiring high-resolution, cross-sectional imaging.

### RELATED APPLICATIONS

This application claims the benefit under 35 USC 119(e) of U.S. Provisional Application No. 63/600,099, filed on Nov. 17, 2023, and U.S. Provisional Application No. 63/652,442, filed on May 28, 2024, both of which are incorporated herein by reference in their entirety.

### BACKGROUND OF THE INVENTION

Optical coherence tomography (OCT) is a cross-sectional, non-invasive imaging modality that is used in many areas of medical imaging. For example, in ophthalmology, OCT has been widely used for imaging the retina, choroid and anterior segment. Functional imaging of the blood velocity and vessel microvasculature is also possible.

Fourier-domain OCT (FD-OCT) has recently attracted more attention because of its high sensitivity and imaging speed compared to time-domain OCT (TD-OCT), which uses an optical delay line for mechanical depth scanning with a relatively slow imaging speed. The spectral information discrimination in FD-OCT is accomplished either by using a dispersive spectrometer in the detection arm (spectral domain or SD-OCT) or rapidly scanning a swept laser source (swept-source OCT or SS-OCT).

Compared to spectrometer-based FD-OCT, swept-source OCT (SS-OCT) has several advantages, including its robustness to motion artifacts and fringe washout, lower sensitivity roll-off and higher detection efficiency.

Many different high-speed swept source architectures have been proposed for SS-OCT. One approach employs a semiconductor optical amplifier (SOA) based ring laser design (see for example Yun et al. “High-speed optical frequency-domain imaging” Opt. Express 11:2953 2003 and Huber et al. “Buffered Fourier domain mode locking: unidirectional swept laser sources for optical coherence tomography imaging at 370,000 lines/s,” Opt. Lett. 31:2975-2977, 2006). Short cavity lasers (see for example Kuznetsov et al. “Compact Ultrafast Reflective Fabry-Perot Tunable Lasers For OCT Imaging Applications,” Proc. SPIE 7554:75541F 2010) are another example. SOA based ring laser designs have been practically limited to positive wavelength sweeps (increasing wavelength) because of the significant power loss that occurs in negative tuning. This has been attributed to four-wave mixing (FWM) in SOAs causing a negative frequency shift in intracavity light as it propagates through the SOA (Bilenca et al. “Numerical study of wavelength-swept semiconductor ring lasers: the role of refractive-index nonlinearities in semiconductor optical amplifiers and implications for biomedical imaging applications,” Opt. Lett. 31:760-762 2006).

At the same time, other architectures exist for SS-OCT that reduce the performance requirements for the swept laser source. Fechtig et al. in an article entitled “Line-Field parallel swept source MHz OCT for structural and functional retinal imaging”, Biomedical Optics Express 716, Vol. 6, No. 3, (2015) describes a system that achieves 1 MHz equivalent A-scan rates by combining a lower sweep rate laser with a linear sensor. Even earlier examples exist such as “Line-Field Optical Coherence Tomography Using Frequency-Sweeping Source” by Lee et al. in IEEE Journal of Selected Topics in Quantum Electronics, Vol. 14, No. 1, January 2008.

### SUMMARY OF THE INVENTION

A cat's-eye tunable laser free space coupled to an interferometer in a manufacturable line-field swept source OCT system.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is schematic side views of a cat's-eye tunable laser free space coupled to an interferometer in a line-field swept source OCT system;

FIG. 2 is a top view of the interferometer according to the current embodiment;

FIG. 3 is a picture of the interferometer according to the current embodiment;

FIG. 4A shows the beam profile for the cat's-eye tunable laser, FIG. 4B is a plot of power as a function of position laterally across the beam, and FIG. 4C is a plot of power as a function of position vertically across the beam;

FIG. 5 is a plot of laser power along the major axis of the line as rendered on the retina 204 of the patient's eye 202;

FIG. 6 is a perspective view of the OCT system;

FIG. 7 is a perspective view of the OCT system with the cowling removed; and

FIG. 8 is a perspective view of the OCT system showing the electronics side of the bench.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

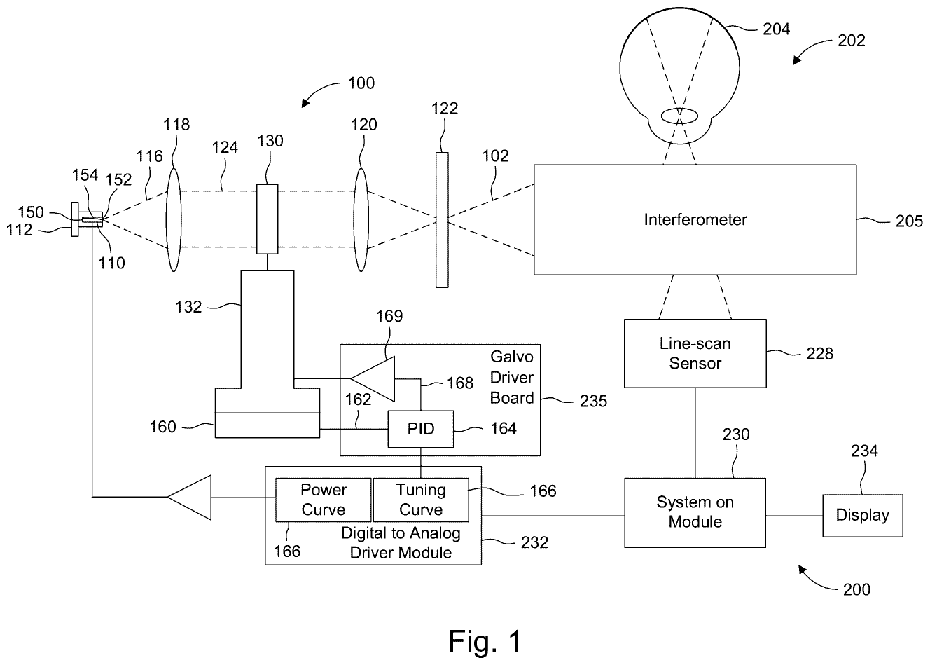

FIG. 1 shows a line-field parallel swept OCT system 200 with a cat's-eye tunable laser 100 coupled to a line-scan or line-field sensor 228 via interferometer 205.

The laser's amplification is provided by a GaAlAs gain chip 110, in one example. The gain chip 110 amplifies light in the wavelength range of about 800 to 900 nanometers. Preferably its center wavelength is around 840 nanometers, which is useful for applications such as ophthalmic imaging and other diagnostic uses because of the water window (650 to 950 nm) at these wavelengths. Another advantage of this wavelength range is that it can be detected with standard cameras with silicon-based imager chips. Specifically, the output is detected with silicon, e.g., complementary metal-oxide-semiconductor (CMOS) or charge-coupled device (CCD), imagers.

In the preferred current embodiment, the gain chip 110 is mounted in a TO-can type hermetic package 112. This protects the chip 110 from dust and the ambient environment including moisture. In some examples, the TO-can package has an integrated or a separate thermoelectric cooler 114.

The chip 110 is preferably a single angled facet (SAF) edge-emitting chip. As such, it has a high reflectivity (HR) coated rear facet 150. It has an antireflective (AR) coated front facet 152. In addition, for improved performance, it has a curved ridge waveguide 154 that is perpendicular to the rear facet 150 but is angled at the interface with the front facet 152. This angling at the front facet along with the AR coating reduces reflections at the front facet reflectivity by up to 40 dB and significantly improves laser performance by reducing parasitic reflections that can otherwise lead to non-smooth tuning and mode-hopping.

The free space beam 116 from the package 112 is diverging in both axes (x, y). It is collimated by a collimating lens 118. The resulting collimated beam 124 is received by a cat's eye focusing lens 120, which focuses the light onto a cat's eye mirror/output coupler 122. This defines the other end of the laser cavity, extending between the mirror/output coupler 122 and the back/reflective facet of the gain chip 110.

The collimated light 124 between the collimating lens 118 and the cat's eye focusing lens 120 passes through a thin film interference bandpass filter 130. This provides a pass band of approximately 0.3 nanometers (nm) full width at half maximum (FWHM) for OCT applications. More generally, its pass band is between 0.2 nm and 0.5 nm FWHM, or more generally between 0.1 nm and 2 nm FWHM. Even more generally, it is between 0.05 nm to 5 nm FWHM.

The bandpass filter is held on an arm of an angle control actuator 132 that changes the angle of the bandpass filter 130 to the collimated light 124. Generally, the angle is modulated over a range of greater than 10 degrees and typically greater than 20 degrees. Currently, the angle is changed between about 110 degrees to about 130-140 degrees or more, measured between the plane of the filter 130 and the axis of the beam 124. In one example, the angle control actuator is a galvanometer. In other examples, the angle control actuator 132 is a servomotor or an electrical motor that continuously spins the bandpass filter 130 in the collimated beam 124. This allows for tilting of the bandpass filter 130 with respect to the collimated beam 124 to thereby tilt-tune the filter and thus change the passband to scan or sweep the wavelength of the swept laser 100.

Tuning speed specifications for a galvanometer generally range from 0.1 Hz to 50 KHz. For the higher speeds, a resonant galvanometer can be used with bi-directional tuning such as a galvanometer operating at greater than 1 kHz to 25 kHz or higher, but higher and lower speeds can be used. Wavelength tuning speed is usually given in nm/sec, so for a 100 Hz tuning speed ideal for retinal imaging applications where a line-speed camera at 100 kHz will give 1000 sampled bandwidth points and 70 nm tuning range, this would give 70 nm/10 msec=7000 nm/sec. In general, the tuning speed should be between 3,000 nm/sec and 11,000 nm/sec or higher.

For retinal or industrial imaging with low-cost CMOS or CCD cameras, 840 nm center wavelength is an ideal water window. The tuning range is usually minimally 30 nm of tuning range. Preferably, the tuning range is closer to 60 nm or 70 nm or more. This provides good resolution of <8 micrometers in air. In general, the tuning range should be between 30 nm and 100 nm.

The size of the collimated beam 124 is important for many applications. As a general rule, a smaller beam results in higher divergence resulting in a larger cone half angle (CHA). This reduces the minimum line width over angle for a tunable filter. In the current embodiment, the collimated beam is preferably not less than, i.e., greater than, 1 millimeter (mm) FWHM and is preferably greater than 2 mm FWHM for retinal OCT application. It can be smaller, however, for many spectroscopy applications in the infrared, visible or ultraviolet. In general, the CHA should be less than .04×.02 degrees and preferably about .02×.01 degrees or less.

The light from the gain chip is polarized. In the common architectures, the polarization is horizontal or parallel to the epitaxial layers of the edge-emitting gain chip 110. In the one configuration, the filter is oriented to receive the S polarization in order to maintain narrow line width of the filter as it is tilt tuned. On the other hand, the P polarization broadens somewhat at large tilt angles. S polarization has higher loss at larger tilt angles than P. So, the filter design needs to address these issues by providing a low enough loss across the tuning band for S, in the current embodiment. However, in some examples, the P polarization is used to provide higher power.

In general, the present cat's-eye configuration provides a number of advantages. It provides low loss, low tolerance, repeatable stable operation since it provides for a lower angle wavelength change over grating-based lasers.

The mirror/output coupler 122 will typically reflect about 80% of the light back into the laser's cavity and transmit about 20% of light. More generally, the mirror/output coupler can reflect from 10% to 99% of light (transmitting 90% to 1%, respectively), depending on the output power and laser cavity loss desired. Higher reflectivity results in lower loss cavities and thus wider laser tuning range where gain exceeds loss, but results in lower output power. In typical operation, the mirror/output coupler 122 reflects less than 90%.

Here, the diverging beam 102 from the mirror output coupler 122 is sent to the interferometer 205.

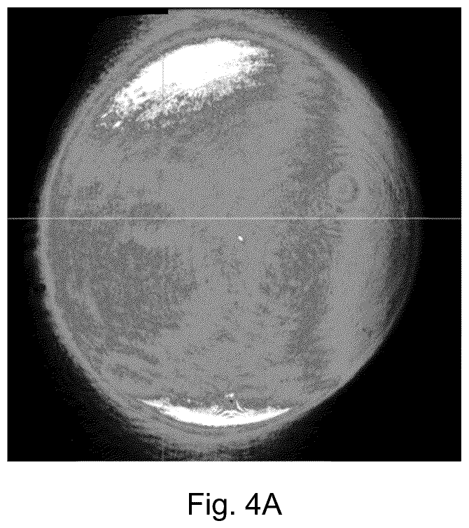

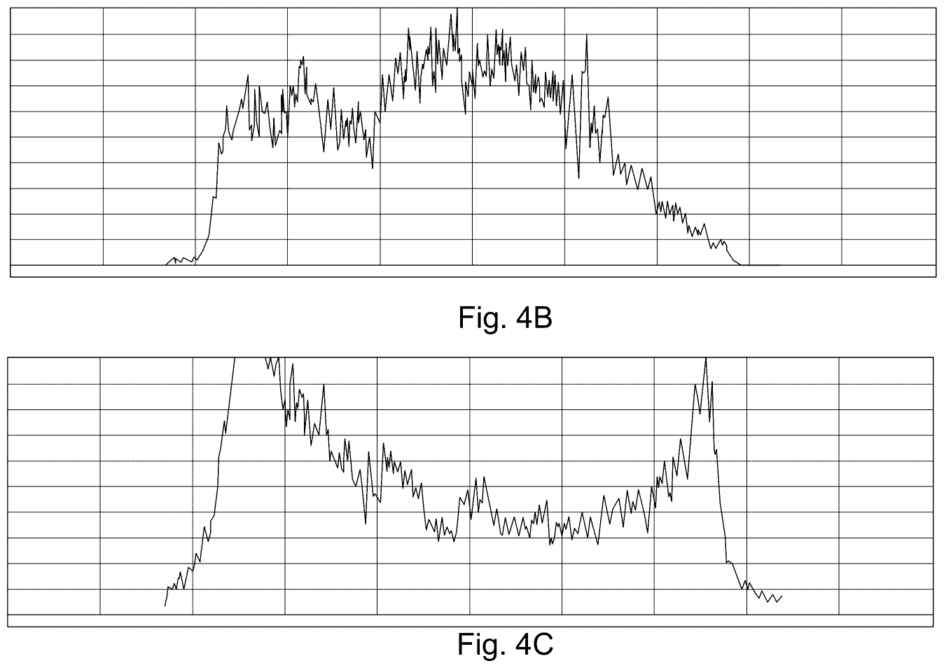

One characteristic of the beam 102 from the laser 100 is that it exhibits higher order spatial modes. FIG. 4 shows exemplary beam profile information. Typically, such modes, even if present in the laser's cavity, are stripped out by intervening single mode fiber. However, in the current embodiment, these higher order modes in the beam are preserved by the free space coupling between the laser 100 and the interferometer 205. In this way, the typically Gaussian power roll off associated with single spatial mode beams is avoided or at least partially mitigated. Instead, the present system provides a more top hat beam profile or a more consistent power profile across the extent of the beam 102 and most importantly along the line that will be projected on the patient's retina.

The angle control actuator 132 is operated as a servomechanism. In the illustrated embodiment, the angle control actuator 132 is a servo controlled galvanometer with an encoder 160. The encoder 160 produces an angle signal 162 indicating the angle of the galvanometer and thus the filter 130 to the collimated beam 124. Preferably, the encoder is an optical encoder and is often analog.

The galvanometer 132 is operated by a galvo driver board 235 that receives the angle signal 162. A PID (proportional-integral-derivative) controller 164 is implemented on the galvo driver board 235 that compares the instantaneous angle signal 162 to a desired angle dictated by a tuning curve. The PID controller 164 produces the control function 168 that is used to drive the windings of the galvanometer 132 via an amplifier 169.

In the illustrated example, the OCT system 200 is employed for ophthalmic analysis of a human eye 202 and specifically the retina 204. That said, the system can also be used for analysis of other samples, both living and non-living, including industrial uses.

Light in the form of free space beam 102 from the laser 100 passes to interferometer 205 that couples light between line-scan sensor 228 and the sample 202 such as a patient's eye 202.

The line field sensor typically has a linear array of at least 512 pixels, and often at least 1024 or 2048 pixels to detect interference signals for a line. In a current example, the linear array is a few pixels wide such as between 2 and 10 pixels wide. Often the pixels can be binned in this lateral axis for higher sensitivity.

In the current implementation, the OCT system 200 is controlled by a single board computer 230. Specifically, it is System on Module (SOM) that includes a graphic processing unit (GPU), central processing unit (CPU), memory, power management, high-speed interfaces. Currently a Jetson Orin series module is used from NVIDIA Corporation.

The SOM 230 controls a digital to analog driver module 232, which principally controls the drive to the chip 110 and the angle control actuator/galvanometer 132. In more detail, the digital to analog driver module 232 includes a tuning curve module that stores a specified tuning function for the angle of the filter 130. This is supplied to the PID controller 164, which tries to minimize the error between the angle signal 162 and the tuning curve across the wavelength sweep of the laser 100. Often, the desired tuning curve is stored in a look up table or is generated algorithmically. Often this is an approximately sawtooth or triangular waveform.

The output from the line field sensor 228 is readout by SOM 230. The results can be stored in the SOM 230 and/or displayed on display 234. The Fourier transform of the interference light performed by the GPU within the SOM 230 at the different wavelengths or frequencies of the swept laser 100 reveals the profile of scattering intensities at different path lengths, and therefore scattering as a function of depth (z-direction) in the sample (see for example Leitgeb et al., “Ultrahigh resolution Fourier domain optical coherence tomography,” Optics Express 12(10):2156 2004). The profile of scattering as a function of depth for a point is called an axial scan (A-scan). The combination of the projected line and line-scan sensor 228 produces a cross-sectional image (tomogram or B-scan) of the sample.

In one implementation, the SOM runs OCTproZ, which is open source software for optical coherence tomography (OCT) processing and visualization available github.com/spectralcode/OCTproZ. A plug-in system enables the integration of custom OCT systems and software modules.

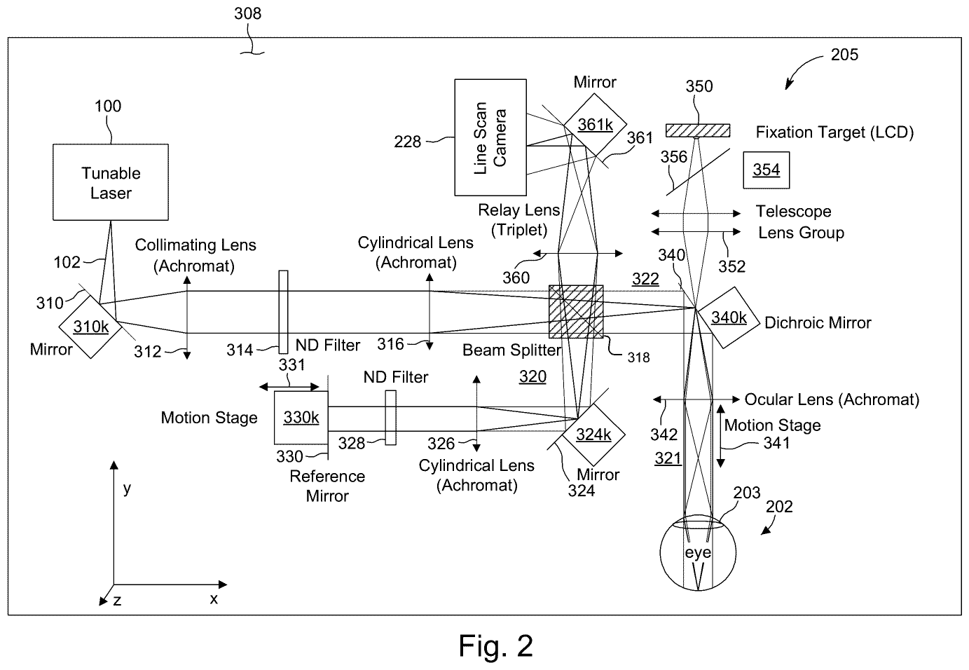

FIG. 2 shows the details of the interferometer 205 and its interfacing with the tunable laser 100 and line scan camera 228.

The free space beam 102 from the laser 100 is diverging. It is received by a mirror 310 mounted on a kinematic mount, which is turn is mounted to a bench 308. The kinematic mount 310K minimally provides for adjusting the direction of the light in both elevation and azimuth, relative to the plane of the bench 308. In some examples, the kinematic mount 310K provides for adjusting the direction of the light along with positionally by provide for fine adjustment of its position in along the x and y axes. This enables alignment of the beam for subsequent optics.

A series of components function as line-forming optics. They convert the light from the laser 100 into a line or more specifically a rectangular profile with an aspect ratio of at least 10 to 1 and typically greater than 100:1, and often 400:1, or more, measured at FWHM. That is, when looking along its optical axis, the light from the line-forming optics has a line or more specifically a high aspect ratio rectangular two-dimensional profile that is at least 10 times longer in the z-axis direction than along the y-axis, for example, measured at the FWHM.

A collimating lens 312 of the line-forming optics collimates the beam from mirror 310. Preferably collimating lens 312 is an achromat. This achromatic lens is designed to minimize the effects of chromatic aberration across the scan band of the laser 100. Chromatic aberration is a problem that occurs when different wavelengths of light are focused at different points, resulting in a blurry image. Currently, achromatic collimating lenses 312 includes two lenses made of different materials to correct for chromatic aberration over the scan band.

A neutral density filter 314 is provided to lower the power of the beam such as by lowering the power by 50% or more. In other examples, this filter is not necessary.

Next a cylindrical achromat lens 316 is provided to form the beam into a line. In the illustrated example, lens focuses the light in the x-y plane so that the line extends in the direction of the z-axis.

A cube beam splitter 318 next divides the laser light between a reference arm 320 and a sample arm 321.

In the reference arm 320, a reference arm mirror 324 is mounted to the bench 308 via a kinematic mount 324K. The reference arm mirror 324 folds the beam path and also allows for alignment. Next a cylindrical achromatic lens 326 collimates the beam. A reference arm neutral density filter 328 adjusts the power of the reference arm light and a reference arm mirror 330 is mounted on a linear motion rail 331 which in turn mounted to the bench 308. The reference arm mirror 330 is moved on the linear motion rail 331 extending in the direction of the x axis and moved by a linear motion actuator 331A to define and control the end of the reference arm and thus control of the delay to path match to the sample 202. Preferably the reference arm mirror 330 is mounted to the linear motion rail 331 via a kinematic mount 330K.

In the sample arm 321, a sample arm dichroic mirror 340 is held on a kinematic mount 340K, which is mounted on the bench 308. It folds the beam path and also allows for alignment by adjustment of its kinematic mount 340K in both azimuth and elevation. Light from a fixation target display 350 and to alignment camera 354 are transmitted through the dichroic mirror 340.

A telescope lens group 352 locates the fixation target 350 at infinity from the perspective of the patient's eye and its focus. The dichroic mirror 340 allows the green fixation target light to be transmitted to the patient and visible light from the patient to be transmitted to the alignment camera 354. Its advantage versus a long-pass dichroic is that the OCT beam is reflected instead of transmitted, which should avoid self-coherence and/or multireflection into the system. A 50/50 beamsplitter 356 couples light to the camera while transmitting light from the target to the patient.

In the sample arm, an achromat ocular lens 342 conditions the light so that the line is in focus on the retina, in conjunction with the optical power of the eye's lens 203. The achromat ocular lens 342 is installed on a linear motion rail 341 and moved by a linear motion actuator 341A to adjust the len's position along the y-axis based on the patient's refractive error.

The light from the reference arm 320 and the sample arm 321 is combined in beamsplitter 318 and directed to the line scan or line field sensor 228. The linear array of the sensor 228 extends in the z-axis direction. A relay lens 360 is currently a triplet. This triplet is a Steinheil Triplet specifically, because it, with a single lens, provides a finite conjugate with relatively good aberration performance. A camera mirror 361 is mounted on a kinematic mount 361K to enable alignment of the interference beam to the line-scan camera 228.

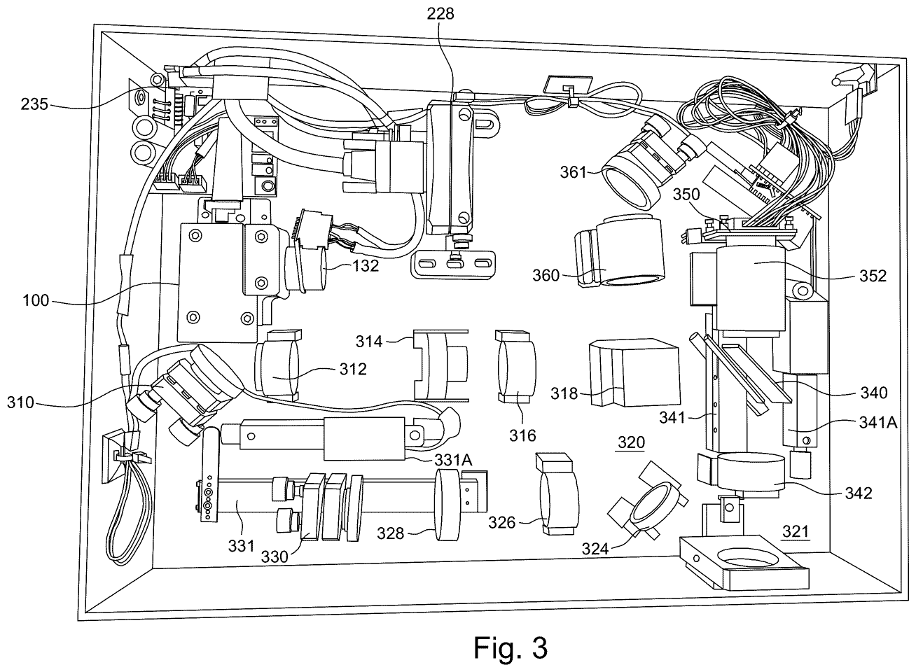

FIG. 3 is a photograph of the line-field parallel swept OCT system 200.

FIGS. 4A, 4B, and 4C show the beam profile of the light from the laser 100 prior to formation into a line. The light exhibits two hot spots, one near the top and one near the bottom of the beam. At the same time, the beam is somewhat cold on the right hand side. The remainder of the beam shows some uniformity. In any event, the profile demonstrates that the beam is it combination of multiple spatial modes. It is believed that this multi spatial mode beam arises out of a number of factors. First, the SAF chip is weakly guiding combined with the fact that the laser cavity can support multiple spatial modes. Further, these spatial modes, due to the absence of any fiber in the system, ensures that the beam is a superposition of the fundamental and several higher order modes.

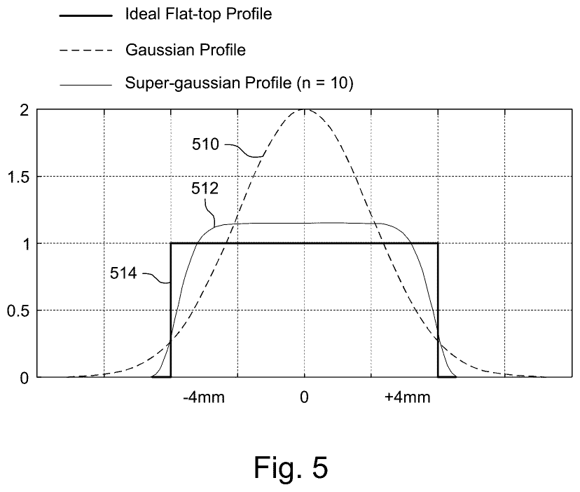

FIG. 5 is a plot of laser power along the major axis of the line as rendered on the retina 204 of the patient's eye 202 and thus after being formed into a line beginning with cylindrical achromat lens 316. In a preferred embodiment, this line is at least 5 millimeters (mm) long, but is preferably longer such as 6 mm or more or 8 millimeters as shown at the back of the eye 202.

In the case of the single spatial mode beam, the line would exhibit a Gaussian profile. This is suboptimal since at the center of the line there would be concern that optical power would exceed safety limits while insufficient power is provided near the edges at −/+4 mm for adequate signal to noise in the images.

In contrast, the present system includes a laser that generates a beam comprising several spatial modes, such as 2 or 3 spatial modes. For example, in one embodiment, the beam comprises 2 or 3 spatial modes in the lateral direction. In another embodiment, the beam comprises 2 or 3 spatial modes in the transverse direction. However, in current preferred embodiment, the beam comprises 2 or 3 spatial modes in both lateral and transverse directions. As a result of these multiple spatial modes, the power distribution has a super Gaussian profile 512 along the extend of the line as projected onto the retina of the eye 202. Thus, the line better approximates the ideal flat-top profile 514.

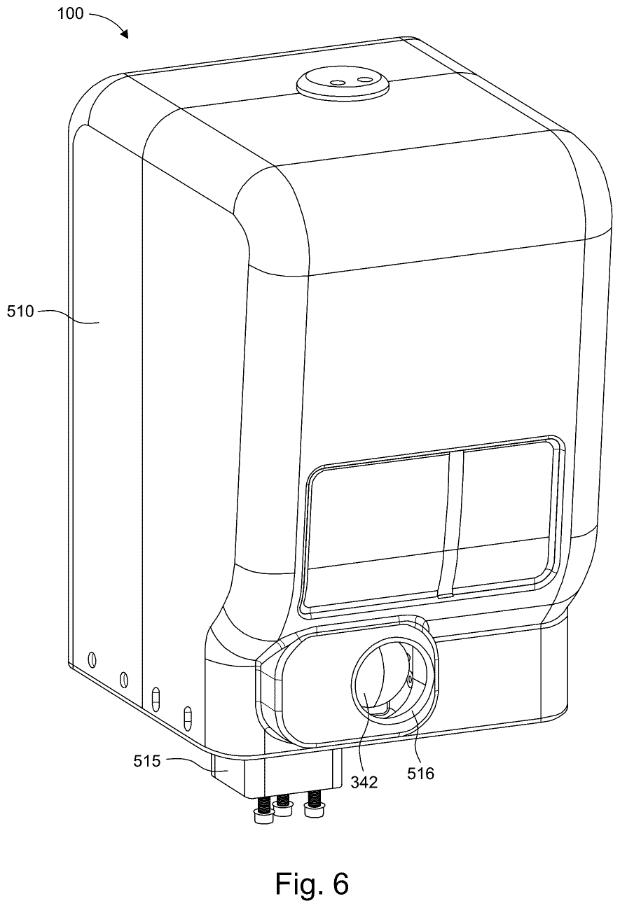

FIG. 6 is a perspective view of the OCT system. It includes an outer two piece cowling 510. A bottom mounting bracket 515 enables mounting on a chinrest. A front port 516 in the cowling 510 exposes the ocular lens 342.

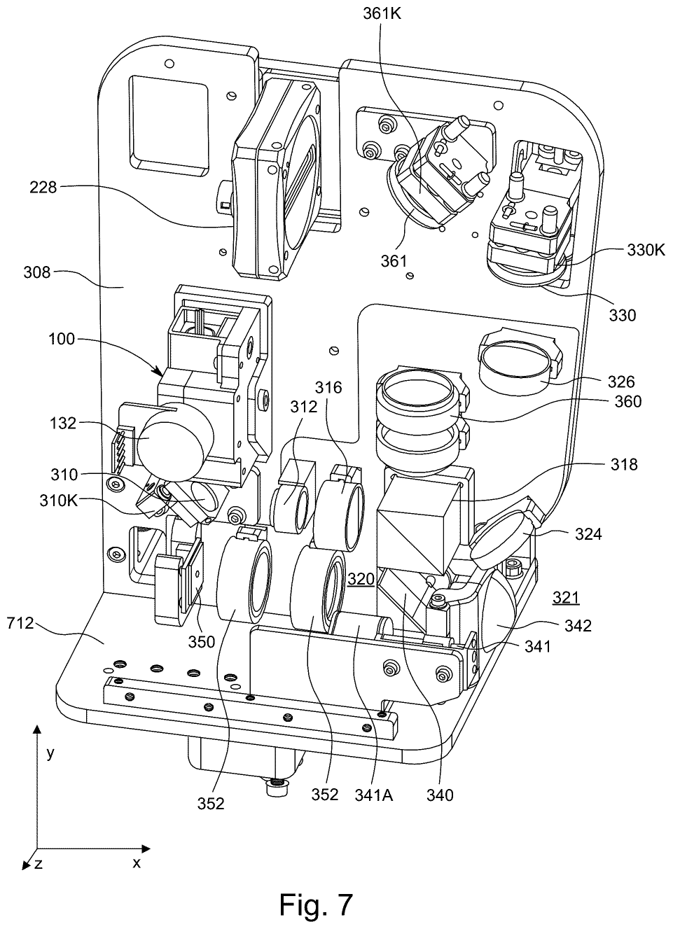

FIG. 7 is a perspective view of the OCT system with the cowling removed. Its optical train is generally as described with reference to FIG. 2 with the same reference numerals being used to show the correspondence. It does have additional functionality. Specifically, the dichroic mirror 340 also functions as a scanning mirror that sweeps the line across the retina in a direction orthogonal to the major axis of that line. This performs a ‘paintbrush’ scan of the retina by sweeping the line vertically by rotating the dichroic mirror around an axis that is parallel to the z axis.

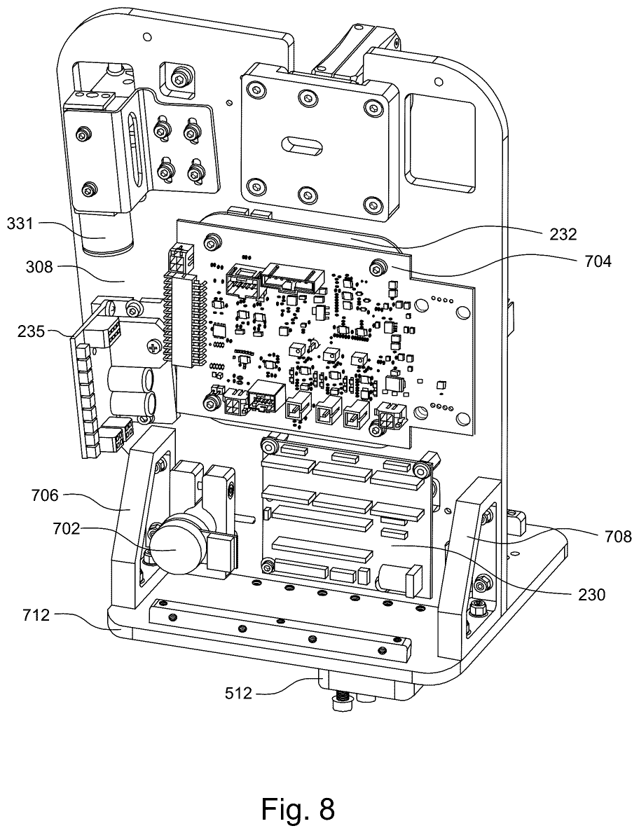

FIG. 8 is a perspective view of the OCT system showing the electronics mounted to the backside of the bench.

It shows a scanning galvanometer 702 for rotating the dichroic mirror 340 for creating the paintbrush scan.

It also shows the highly integrated nature of the present system. The optical train is installed on one side of the bench 308 whereas the electronics are generally installed on the other side.

In more detail, the single board computer 230 is mounted near the bottom of the bench. The digital to analog driver module 232 which principally controls the drive to the chip 110 and the angle control actuator/galvanometer 132 along with the scanning galvanometer 702. Also shown in an analog board 704 on which the chip injection current driver is provided.

Two angle brackets 706 and 708 connect the bench 308 to a bottom plate 712 that connects to the bottom mounting bracket 512.

In operation, the laser 100 is swept in wavelength so that its frequency changes preferably linearly with time over the sweep or B-scan period. A linear frequency sweep is often desirable because it allows for efficient use of the camera sample rate and avoids the need to resample.

For the linear sweep in frequency, the angle of the filter 130 must be tuned in a non-linear fashion. As shown, the rate of change of the angle of the filter 130 slows with increasing angle and shorter wavelengths, higher frequency of the laser emission 102. This tuning curve for linear frequency sweeping is produced by and/or stored in the tuning curve module 166.

As the laser 100 begins its sweep, a trigger signal initiates the capturing of the line interference signals as the laser tunes. Preferably, at least 250 line interference signals are captured by the line-scan camera 228 within the sweep period of the laser 100. Currently more than 500 line interference signals, such as 1000 line interference signals or more are captured.

Preferably, the sweep period is less than 0.05 seconds and preferably than 0.02 seconds. In the current examples, a sweep period of about 0.01 seconds or less provides acceptable B-scans of the human eye despite microsaccades and other movement.

While this invention has been particularly shown and described with references to preferred embodiments thereof, it will be understood by those skilled in the art that various changes in form and details may be made therein without departing from the scope of the invention encompassed by the appended claims.

What is claimed is:

1. An optical coherence tomography (OCT) system comprising:
a swept laser source configured to generate a beam comprising multiple spatial modes in at least one of a transverse or lateral direction;
an interferometer free-space coupled to the swept laser source, the interferometer configured to receive the beam comprising multiple spatial modes and to form it into a line-shaped illumination projected onto a sample, wherein the free-space coupling preserves the multiple spatial modes of the beam;
a line-field detector configured to detect interference signals resulting from the illumination of the sample with the line-shaped beam; and
wherein the preservation of the multiple spatial modes in the beam results in a line-shaped illumination with a more uniform intensity profile across its length when projected onto the sample, enhancing imaging performance by mitigating Gaussian power roll-off associated with single spatial mode beams.

2. The OCT system of claim 1, wherein the interferometer includes line-forming optics comprising at least one cylindrical lens configured to form the beam into the line-shaped illumination with an aspect ratio of at least 10:1 measured at full width at half maximum (FWHM).

3. The OCT system of claim 1, wherein the line-shaped illumination projected onto the sample has a length of at least 5 millimeters when projected onto the sample surface.

4. The OCT system of claim 1, wherein the line-field detector comprises a linear array of pixels configured to capture at least 500 line interference signals during each sweep period of the swept laser source.

5. An optical coherence tomography (OCT) system comprising:
a swept laser source configured to generate a line-shaped illumination beam;
an interferometer coupled to the swept laser source, the interferometer configured to direct the line-shaped illumination beam toward an eye;
a scanning dichroic mirror positioned in the sample arm of the interferometer, the scanning dichroic mirror configured to:
reflect the line-shaped illumination beam toward the sample while rotating around an axis parallel to the length of the line-shaped beam to scan the beam across the eye in a direction orthogonal to its length, thereby performing a paintbrush scan; and
transmit light from a fixation target display through the scanning dichroic mirror toward the eye, allowing a user to view the fixation target during scanning;
a line-field detector configured to detect interference signals resulting from the illumination of the eye with the scanned line-shaped beam;
wherein the scanning dichroic mirror enables simultaneous paintbrush scanning of the line-shaped beam and transmission of the fixation target to the user, enhancing imaging performance and user alignment in the OCT system.

6. The OCT system of claim 5, wherein the scanning dichroic mirror is mounted on a galvanometer configured to rotate the mirror at a controlled rate, enabling precise scanning of the line-shaped beam across the eye in synchronization with the swept laser source.

7. The OCT system of claim 5, further comprising a fixation target display configured to present a visual target at optical infinity, assisting the user in maintaining a steady gaze during the scanning process.

8. An optical coherence tomography (OCT) system comprising:
a swept laser source configured to generate a line-shaped illumination beam;
an interferometer coupled to the swept laser source, the interferometer configured to direct the line-shaped illumination beam toward a sample;
a line-field detector configured to detect interference signals resulting from the illumination of the sample with the line-shaped beam;
an integrated computer within the OCT system, the integrated computer configured to:
control the operation of the swept laser source and the line-field detector;
store a tuning function for adjusting an angle of a filter within the swept laser source to achieve linear frequency sweeping;
process the interference signals detected by the line-field detector to generate cross-sectional images of the sample;
wherein the integration of the computer within the OCT system enables real-time control and processing, enhancing imaging performance and system compactness.

9. The OCT system of claim 8, wherein the integrated computer comprises a single-board computer with an integrated graphics processing unit (GPU) configured to perform real-time Fourier transforms on the interference signals.

10. The OCT system of claim 8, wherein the integrated computer is configured to run open-source OCT processing software that allows for integration of custom OCT systems and software modules.

11. The OCT system of claim 8, further comprising a display connected to the integrated computer, the display configured to present the generated cross-sectional images to a user in real-time.

12. The OCT system of claim 8, wherein the integrated computer is configured to synchronize the operation of the swept laser source with the line-field detector to capture at least 500 line interference signals during each sweep period of the swept laser source.

13. The OCT system of claim 8, wherein the computer is a signal board computer.

14. The OCT system of claim 8, wherein the computer is mounted to one side of a bench and optics of the interferometer are installed on the other side of the bench.

15. The OCT system of claim 14, further comprising a bottom plate for supporting the bench.
